(19) United States
(12) Patent Application Publication
(10) Pub. No.: US 2011/0135747 A1
(43) Pub. Date: Jun. 9, 2011

(54) HOMEOPATHIC THERAPEUTIC METHOD

(76) Inventor: Nancy Josephine Polich, Naperville, IL (US)

(21) Appl. No.: 13/057,495

(22) PCT Filed: Aug. 28, 2009
(86) PCT No.: PCT/US2009/055456

§ 371 Date: Feb. 4, 2011

Related U.S. Application Data
(60) Provisional application No. 61/093,126, filed on Aug. 29, 2008.

(51) Int. Cl. A61K 35/64; A61K 36/28; A61K 33/42; A61K 36/38; A61K 36/45; A61K 36/22; A61K 36/75; A61K 36/30; A61K 36/81; A61K 33/26; A61K 33/06; A61K 36/42; A61K 33/04; A61K 33/10; A61P 19/04; A61P 17/02; A61P 17/00

(52) U.S. Cl. 424/539; 424/725; 424/602; 424/730; 424/538; 424/646; 424/696; 424/709; 424/687

(57) ABSTRACT

Provided are methods of topical application of an aqueous homeopathic composition for treating various ailments and injuries, including deep tissue wounds and surgical wounds. The aqueous homeopathic composition is formulated in a high potency of at least 400 C, maintained in an aqueous medium and topically applied to a bodily surface for an extended period of time to render therapeutic treatment.

### CROSS REFERENCE TO RELATED APPLICATIONS

This application is a non-provisional of and claims priority to U.S. Provisional Application No. 61/093,126, filed Aug. 29, 2008, under 35 U.S.C. 119(e), the entire disclosure of which is incorporated herein by reference.

### FIELD OF THE INVENTION

The invention is related to therapeutic methods for treating injuries or ailments, including but not limited to, tissue damage, such as surgical wounds and deep tissue wounds, skin diseases, pain, inflammation and infection, using a topically administered, high potency, homeopathic composition.

### BACKGROUND OF THE INVENTION

Homeopathy is a medical practice that treats a disease, affliction or condition by administering small amounts of a natural substance. A fundamental aspect of homeopathy is the stimulation of the body's natural healing process by administering a homeopathic remedy capable of producing the same or similar symptoms for which treatment is sought in a healthy individual. For example, arnica, which causes bruising in a healthy individual when administered in concentrated dosages, is a common homeopathic remedy for treating bruising.

In the United States, the preparation and sale of homeopathic remedies are regulated by the Homeopathic Pharmacopoeia of the United States (HPUS). According to HPUS guidelines, homeopathic remedies are prepared by successively diluting herbals and other natural substances in a water and/or alcohol solution. Because homeopathic potency is inversely related to concentration, the greater the dilution the higher the potency of the homeopathic remedy.

The most common methods for administering homeopathic remedies involve sipping a water based solution or orally dissolving a homeopathic composition infused sucrose/lactose pellet in the mouth. It is well recognized that certain orally administered homeopathic remedies may be formulated as high potency compositions to enhance therapeutic effectiveness. While these oral administrations are effective for treating certain systemic diseases, they are generally inadequate for treating some localized ailments because: oral formulations cannot be directly administered to location of ailment; the administration area is limited; and therapy is likely limited to less than about 20 minutes due to the natural cleansing process of the mouth, for a typical number 40 pellet. Furthermore, studies of oral homeopathic administration have shown no to minimal efficacy for the treatment of localized ailments.

While topical gel and cream based homeopathic remedies may be directly administered to the site of an ailment, they are formulated in lower potencies, generally about 6× or less, because the prior art teaches that efficacy of topical homeopathic formulations diminishes as potency increases. This teaching is so prevalent and widely accepted that most homeopathic gels and creams have a potency less than about 3×, and it is extremely rare to find a topical homeopathic composition in potencies greater than 6×. The low potency of topical homeopathic compositions significantly limits their use to only minor ailments. For more serious localized ailments, homeopaths generally recommend and administer oral homeopathic remedies having potencies greater than 6×.

Although some references suggest that it may be possible to form high potency topical homeopathic compositions, their effectiveness is questionable as these same references also teach that high potency oral compositions are preferred and more therapeutically effective. Without wishing to be bound by theory, it is believed that these anecdotal high potency topical compositions are ineffective in part because the homeopathic composition is not maintained in an aqueous carrier and held in contact with a bodily surface for an extended period of time. Irrespective of anecdotal suggestions that high potency topical compositions may be formulated, there is no clear prior art teaching that high potency topical formulations would be effective for treating any injury or ailment, particularly severe traumas, such as surgical or deep tissue wounds.

U.S. Pat. No. 7,229,648 (Dreyer), for example, discusses the possibility of formulating a homeopathic gel for treating pain and inflammation having a potency of greater than 400×, such as 1M, 10M and 50M. Dreyer, however, specifically teaches that potencies above 400× will be more effective when used internally rather than topically. (See Table 1, col. 6, lines 61-67). Additionally, Dreyer teaches that its topical gel formulation should be dry to the touch within a few minutes of application (See col. 4, line 55). These conventional gels do not maintain the homeopathic composition in an aqueous environment; consequently, the composition is rapidly absorbed and/or metabolized by the skin. Dreyer therefore provides no mechanism for maintaining the homeopathic composition in an active form in contact with a bodily surface for an extended period of time to be maximally therapeutically effective.

U.S. Patent Application Publication No. 2008/279902 (Luria) discloses a cosmetic composition including a homeopathic complex that may be formulated as a transdermal or topical paste, cream, lotion, ointment or gel having a potency of about 1× to about 50,000 Q or about 100 C to about 50,000 Q. The topical cosmetic composition of Luria, however, does not disclose maintaining the homeopathic complex in an aqueous carrier in contact with a bodily surface or applying the homeopathic complex to the site of an injury for an extended period time to be therapeutically effective.

Therefore, there remains a need to develop an effective topically administered homeopathic therapeutic treatment that may particularly suitable for treating severe ailments and injuries.

### SUMMARY OF THE INVENTION

The present invention pertains to a novel therapeutic homeopathic method. In a first aspect, the invention is directed a therapeutic method comprising the steps of: topically administering a homeopathic composition having a potency of at least 400 C to a bodily surface, and maintaining the homeopathic composition in an aqueous environment and in contact with a bodily surface continuously for at least 30 minutes or intermittently for a total of 2 hours or more in a 24 hour period.

In a second aspect, the therapeutic method comprises the steps of: topically administering an aqueous homeopathic composition having a potency of at least 400 C to a bodily surface, and maintaining the homeopathic composition in an aqueous environment and in contact with the bodily surface continuously for at least 30 minutes or intermittently for a total of 2 hours or more in a 24 hour period.

In a third aspect, the therapeutic method comprising the steps of: topically administering a homeopathic composition having a potency of at least 400 C to a bodily surface, and maintaining the homeopathic composition in an aqueous environment and in contact with the bodily surface for an extended period of time sufficient to effectively treat a deep tissue wound or surgical injury.

### DESCRIPTION OF CERTAIN PREFERRED EMBODIMENTS OF THE INVENTION

For illustrative purposes, the principles of the present invention are described by referencing various exemplary embodiments thereof. Although certain embodiments of the invention are specifically described herein, one of ordinary skill in the art will readily recognize that the same principles are equally applicable to, and can be employed in other apparatuses and methods. Before explaining the disclosed embodiments of the present invention in detail, it is to be understood that the invention is not limited in its application to the details of any particular embodiment shown. The terminology used herein is for the purpose of description and not of limitation. Further, although certain methods are described with reference to certain steps that are presented herein in certain order, in many instances, these steps may be performed in any order as may be appreciated by one skilled in the art, and the methods are not limited to the particular arrangement of steps disclosed herein.

For purposes of the present invention, the term “ailment,” as used herein, refers to any disease, disorder, associated condition thereof, or associated symptom thereof.

As used herein, the term “aqueous homeopathic composition,” refers to a homeopathic composition dissolved in, suspended in or otherwise mixed with any suitable aqueous medium that contains water molecules and is suitable for enabling topical administration.

As used herein, the term “homeopathic composition” refers to a composition formulated from one or more naturally derived substances, such as herbs, that have been diluted. In an exemplary embodiment, the composition may be prepared according to the Homeopathic Pharmacopoeia of the United States (HPUS) standards and procedures, wherein the natural substance is serially diluted and sufficiently succussed to achieve a desired potency using conventional homeopathic potentization methods. Over the years, many variations of the art of homeopathic production have been employed, including varying the succussion to ratio as in the Dunham potencies and using water to succuss the homeopathic composition as in the Finke remedies. In some cases succession is not even employed. For purposes of the present invention, the homeopathic remedies may be formulated according to methods not taught in the HPUS, which may involve the preparation of tinctures, dilutions, infusions, water or alcohol extracts, powdered plasters, decoctions, poultices, or any other methods of preparations.

As used herein, the term “potency” is defined according to the HPUS and may be quantified according to various scales, such as the decimenal X scale, centesimal C scale and quintamillesimal Q scale. In general, a decimenal X scale dilution is half the value of a C scale dilution, and a given dilution on the Q scale is about 2.35 times the value of a C scale dilution. For example, a 1:10 dilution has a potency of 1 decimal (1×), which is equal to a concentration of 10⁻¹. Similarly, a 1:100 dilution has a potency of 1 centesimal (1 C), which may be expressed as a potency of 2× or a concentration of 10⁻², and a potency of 1 millesimal (1M) may be expressed as having a potency of 1000 C, a potency of 2000× or a concentration of 10⁻²⁰⁰⁰. For purposes of the present invention, a homeopathic composition having a 10⁻⁸⁰⁰ concentration is equivalent to a potency of 400 C or 800×, a concentration of 10⁻¹⁰⁰⁰ concentration is equivalent to a potency of 500 C, a concentration of 10⁻²⁰⁰⁰ is equivalent to a potency of 1000 C or 1M, a concentration 10^{-5,000} is equivalent to a potency of 2.5M, a concentration of 10^{-10,000} is equivalent to a potency of 5M, a concentration of 10^{-20,000} is equivalent to a potency of 10M, a concentration of 10^{-50,000} is equivalent to a potency of 25M, a concentration of 10^{-100,000} is equivalent to a potency of 50M, a concentration of 10^{-200,000} is equivalent to a potency of 100M, a concentration of 10^{-500,000} is equivalent to a potency of 250M, a concentration of 10^{-1,000,000} is equivalent to a potency of 500M, a concentration of 10^{-2,000,000} is equivalent to a potency of 1 CM, a concentration of 10^{-5,000,000} is equivalent to a potency of 2.5 CM, a concentration of 10^{-10,000,000} is equivalent to a potency of 5 CM, a concentration of 10^{-20,000,000} is equivalent to a potency of 10 CM.

Therapeutic effect is not determined in the same way as herbals or pharmaceuticals. In general, homeopathic remedies are made in low concentrations, typically, well below the level where the molecular concentration of the active ingredient is important. For this reason, unlike pharmaceuticals, the quantity is not critical. As evidenced in the above examples, potency is inversely related to concentration; consequently, the greater the dilution, the higher the potency of a homeopathic composition. A desired potency may be achieved by making repeated dilutions of a homeopathic composition. For instance, an active drug having a potency of 1 C may be prepared by making to a 1 in 100 dilution of an active drug or a tincture, extract or derivative thereof, e.g. 1 ml of a tincture mixed with 99 ml of a diluent liquid, and then succussed at least 10 times according to known potentization procedures in homeopathy. A potency of 2 C may be prepared by making a 1:100 dilution of an active drug having a potency of 1 C and then succussing at least 10 times. A potency of 3 C may be prepared by making a 1:100 dilution of an active drug having a 2 C potency and then succussing at least 10 times. Where a homeopathic composition includes a combination of ingredients, the designated potency of the composition is the same potency for each individual ingredient. For example, a 10M Arnica and Ledum homeopathic composition indicates that a 10M potency of Arnica and a 10M potency of Ledum.

As used herein, the term “injury” refers to any bodily damage, wound, condition thereof, or any associated symptoms thereof.

As used herein, the term “natural substances” refers to any material that may be obtained from nature, including but not limited to plant or mineral extracts, such as powder extracts or fluids extracts, one or more active compound of a plant or mineral, any parts of a plant or mineral, or a whole plant or mineral, tinctures thereof, and mixtures thereof.

As used herein, the term “treatment” or “treating” refers to any means for producing a beneficial result in an individual affected with an ailment or suffering from an injury, including but not limited to, substantially preventing, substantially reducing the severity of, substantially improving the condition of, substantially expediting the healing of or substantially curing an ailment, an injury, one or more one symptoms, conditions or aspects thereof, or combinations thereof.

As used herein, the term “topical” application or administration refers to the direct administration of a composition onto one or more bodily surfaces, such as any epithelial tissue, including but not limited to skin; mucosa; connective tissue, including cartilage and bone; muscles; and nervous tissue; organs; nerves; brain; arteriol; lymphatic or combinations thereof. In an exemplary embodiment, topical application refers to administration of a composition to the skin or mucous membranes including but not limited to the vagina, anus, throat, eyes and ears. For purposes of the present invention, bodily surfaces to which the composition is topically applied do not include any surfaces of the oral cavity since this is generally considered to be oral administration, which is outside the scope of the present invention. Unless otherwise stated or implied, topical applications or administrations include transdermal administrations.

It must be noted that as used herein and in the appended claims, the singular forms “a”, “an”, and “the” include plural references unless the context clearly dictates otherwise. Thus, for example, reference to “an active agent” includes a plurality of active agents and equivalents thereof known to those skilled in the art, and so forth. As well, the terms “a” (or “an”), “one or more” and “at least one” can be used interchangeably herein. It is also to be noted that the terms “comprising”, “including”, and “having” can be used interchangeably.

The invention is directed to a novel therapeutic method for effectively treating an ailment or injury by topically applying a high potency, aqueous homeopathic composition to a patient for an extended period of time. Specifically, the therapeutic method of the present invention involves the steps of: (1) topically applying a homeopathic composition having a high potency of at least 400 C to a bodily surface of a patient, and (2) maintaining the homeopathic composition in an aqueous environment in contact with the bodily surface for an extended period of time sufficient to provide a beneficial effect. Without wishing to be bound by theory, this unique combination of therapeutic factors, particularly maintaining the homeopathic composition in an aqueous environment use of a high potency composition and topically administering the aqueous homeopathic composition for an extended period of time, provides substantial and unexpected therapeutic benefits The therapeutic method may be used to treat a wide variety of ailments and injuries, including but are not limited to, tissue damage, including deep tissue damage, such as that caused by surgery, superficial wounds, and skin diseases, as well as any condition associated with a severe injury or ailment, pain, inflammation or infection.

### 1. Formulating a High Potency Homeopathic Composition

A high potency formulation of a homeopathic composition including one or more naturally derived ingredients may be selected and used in the method of the present invention. In an exemplary embodiment, the homeopathic composition may comprise known or conventional homeopathic compositions, including any one or more ingredients disclosed in the HPUS; Boericke, William, “Pocket Manual of Homeopathic Materia Medica,” B. Jain, 1995; Hahnemann, Samuel, “Materia Medica Pura,” 1830; or Schroyens, Frederik, “Synthesis Repertory 9,” Homeopathic Book Publishers, 2004 herein incorporated by reference. Exemplary homeopathic compositions for use in formulating the compositions of the invention may include homeopathic compositions suitable for treating tissue damage, including deep tissue damage, such as that caused by surgery, superficial wounds, and skin diseases, as well as any condition associated with a severe injury or ailment, pain, inflammation or infection. Tables 1-3 provides a list of exemplary compositions and/or ingredients that are particularly effective for treating localized ailments or injuries, any one of which or combinations thereof, may be used to formulate the homeopathic composition of the present invention. In an exemplary embodiment, the homeopathic composition may also include any of the ingredients or combinations thereof listed in Table 4.

In exemplary embodiments, the homeopathic composition may include one or more, two or more, three or more or four or more ingredients selected from Arnica Montana (Arnica), Bellis perennis, Calendula, Hypericum perforatum (Hypericum), Ledum palustre (Ledum), Ruta graveolens (Ruta), Symphytum officinale, Rhus toxicodendron (Rhus Tox), Staphasagria, or Natrum sulphericum. In other exemplary embodiments, the homeopathic composition may include a combination of only Arnica montana, Ledum palustre, Ruta graveolens, and Rhus toxicodendron. Alternatively, the homeopathic composition may include a combination of only Arnica montana and Ledum palustre. In still other exemplary embodiments, the homeopathic composition may include a combination of only Arnica montana, Ledum palustre, Ruta graveolens, Rhus toxicodendron and Hypericum perforatum.

TABLE 1
Injuries

| | |
|---|---|
| GENERALS INJURIES | ARN., CALEN., CAMPH., CANN I., CON., HEP., |
| | HYPER., PULS., RHUS T., RUTA., SUL AC., |
| | SYMPH. |
| GENERALS INJURIES operation - | STAPH. |
| Wound | |
| GENERALS INJURIES overexertion, | BELL P., CALC. |
| strain | |
| GENERALS INJURIES concussion | ARN., BAD., HYPER., NAT S. |
| GENERALS INJURIES contusion | RUTA |
| GENERALS INJURIES Bones; | CARB AC., HYPER., RUTA, SPIG. |
| fractures of | |
| HEAD INFLAMMATION Brain | BELL. |
| HEAD INJURIES of the head; | ARN., NAT S |
| HEAD INFLAMMATION Periosteum | FL AC., MEZ., PH AC. |
| HEAD INFLAMMATION Brain | ACON., ARN., BELL., HYPER., NAT S., SIL. |
| HEAD INFLAMMATION Meninges | BELL, HELL., STRAM., ZINC., |
| HEAD PAIN injuries; after mechanical | NAT S. |
| GENERALS SHOCK injuries; | ACON., ARN., CAMPH., DIG., HYPER., LACH., OP., |
| | VERAT. |
| GENERALS INJURIES sprains | AGN., ARN., CALC., LYC., MILL., NAT C., NAT M., |
| | PETR., PHOS., PLAT., RHUS T., RUTA, STRAM. |
| GENERALS INJURIES Periosteum, of | CALC., RUTA, SPONG., SYMPH. |
| GENERALS INJURIES tennis elbow | AGAR., AMBR., RHUS T., |
| GENERALS INJURIES rupture | ARN., CALC., CALEN., NAT C., NAT M., PHOS., |
| | RHUS T. |
| GENERALS INJURIES Nerves | ALL C., ARN., BELL., HELON., HYPER., LED., |
| | MENY., PH AC., PHOS. |
| GENERALS INJURIES Soft parts, of | ARN., CON. |
| GENERALS INJURIES Tendons, of | ACON., AM C., ANAC., APIS, ARN., ARS., ARS I., |
| | BELL., BENZ AC., BRY., CALC P., CALEN., |
| | CANTH., FERR., GUAJ., HEP., IOD., KALI I., |
| | RHOD., RHUS T., RUTA, SIL., SULPH., SYMPH., |
| | THUJ. |

TABLE 2
Acute Ailments

| | |
|---|---|
| SKIN - ERUPTIONS boils | ARN., BELL., HEP., LACH., LYC., MERC., PETR., PSOR., |
| | RHUS T., SULPH. |
| SKIN - ERUPTIONS blisters | ANT C., CAUST., RHUS T. |
| SKIN - DECUBITUS | ARN., CHIN., GRAPH., LACH., PETR., SEP., SIL. |
| FACE - ERUPTIONS acne | AUR., CARB AN., CARB V., CARBN S., CAUST., FL AC., |
| | GRAPH., HEP., KALI AR., KALI BI., KALI BR., LYC., NAT |
| | M., NUX V., PHOS., PULS., RHOD., RHUS T., SARS., SEP., |
| | SIL., SULPH., SYPH., TEUCR., ZINC. |
| Herpes | AGAR., AM C., ANAN., ARS., BAR C., BOV., CALC., CARB |
| | AN., CARB V., DULC., ELAPS, GRAPH., HEP., KALI AR., |
| | KALI BI., KALI C., KALI I., KALI S., LACH., LED., LYC., |
| | MERC., NAT AR., NAT C., NAT M., NAT S., NICC., NIT AC., |
| | PSOR., RHUS T., SEP., SIL., SULPH., THUJ. |
| SKIN ERUPTIONS pustules | ANT T., ARS., RHUS T., STAPH., SULPH. |
| SKIN ERUPTIONS rhus | ANAC., RHUS D. |
| poisoning | |
| SKIN ERUPTIONS impetigo | ANT C., ARUM T. |
| SKIN ERUPTIONS granular | ACON., AGAR., ALUM., AM C., ARS., BELL., BRY., BUFO, |
| | CARB V., CLEM., COCC., CON., DULC., GRAPH., HEP., |
| | IOD., IP., KREOS., LED., MANC., MERC., MERC C., MEZ., |
| | NAT M., NUX V., OP., PAR., PH AC., PHOS., PSOR., PULS., |
| | RHUS T., SARS., STRAM., SULPH., VALER., VINC., ZINC. |
| SKIN ERUPTIONS herpes | IRIS, MERC., MEZ., RAN B., RHUS T. |
| zoster | |

TABLE 3
Chronic Ailments

| | |
|---|---|
| SKIN ERUPTIONS lichen | ACON., AGAR., ALUM., AM M., ANAN., ANT C., |
| | APIS, ARS., ARS I., BELL., BOV., BRY., CALAD., |
| | CASTN V., CIC., COCC., DULC., JUG C., KALI AR., |
| | KREOS., LED., LYC., MANG., MERC., MUR AC., |
| | NAT C., NAT M., PHYT., PLAN., RUMX., SEP., SUL |
| | I., SULPH., TIL. |
| SKIN ERUPTIONS lichen | AGAR., ANAC., ANT C., APIS, ARS., ARS I., CHIN |
| planus | AR., IOD., KALI BI., KALI I., LED., MERC., SARS., |
| | STAPH., SUL I., SULPH., SYPH. |
| SKIN ERUPTIONS pemphigus | LACH. |
| SKIN ERUPTIONS petechiae | ARS., BRY., PHOS., RHUS T. |
| SKIN ERUPTIONS pityriasis | CARB AC., CAUL., DULC., LYC., MEZ., NAT AR., |
| versicolor | PSOR., SEP., SULPH., TELL. |
| SKIN ERUPTIONS psoriasis | ARS I., LYC., PHYT., SEP. |
| SKIN ERUPTIONS ringworm | ANT C., ANT T., APIS, ARS., BAC., BAPT., BAR M. |
| | CALC., CALC I., CHIM., CUPR., DULC., EUP PER., |
| | GRAPH., HEP., JUG R., KALI I., KALI S., LAPPA, |
| | LYC., MEZ., NAT M., OL J., PHYT., PSOR., RAD BR., |
| | RAN B., RHUS T., SEP., SIL., SULPH., TELL., THUJ., |
| | TUB., VIOL T. |
| SKIN ERUPTIONS scaly | ARS., CLEM., KREOS., PHOS., PHYT., SEP. |
| SKIN ERUPTIONS scabies | ARS., CARB V., CARBN S., CAUST., KALI S., PSOR., |
| | SEL., SEP., SULPH. |
| SKIN ERUPTIONS scarlatina | AIL., AM C., APIS, BELL., LACH., LYC., MERC., NIT |
| | AC., RHUS T. |
| SKIN ERUPTIONS suppurating | ANT C., CHAM., GRAPH., LYC., MERC., NIT AC., |
| | PETR., RHUS T., SEP., SIL. |
| SKIN EXCRESCENCES | CALC., CAUST., GRAPH., LYC., NIT AC., STAPH., |
| | THUJ. |
| SKIN EXCRESCENCES | DULC., LACH., MED., MERC C., NAT S., NIT AC., |
| condylomata | PH AC., THUJ. |
| SKIN FORMICATION | COCA, LYC., PH AC., RHOD., RHUS T., SEC., |
| | SULPH., TARENT. |
| HEAD HAIR falling | AUR., BAR C., CARB V., CARBN S., FL AC., GRAPH., |
| | KALI C., KALI S., LACH., LYC., NAT M., NIT AC., |
| | PHOS., SEP., SIL., SULPH., THUJ. |
| RECTUM FISSURE | CHAM., GRAPH., MUR AC., NIT AC., RAT., SEP., |
| | THUJ. |
| RECTUM FISTULA | AUR M., BERB., CALC., CALC P., CARB V., CAUST., |
| | KALI C., NIT AC., SIL |
| RECTUM HEMORRHOIDS | AESC., AGAR., ALOE, ARS., CARB AN., CARB V., |
| | CAUST., COLL., GRAPH., HAM., KALI AR., KALI C., |
| | KALI S., LACH., LYC., MERC I R., MUR AC., NIT |
| | AC., NUX V., PAEON., PHOS., PULS., SEP., SULPH. |
| NOSE EPISTAXIS | ACON., AM C., AMBR., ANT C., ARN., BELL., |
| | BOTH., BOV., CACT., CALC., CALC P., CALC S., |
| | CARB V., CARBN S., CAUST., CHIN., CROC., CROT |
| | H., FERR PIC., HAM., HYOS., IP., KALI I., LACH., |
| | MED. MELI., MERC., MILL., NIT AC., PHOS., PULS., |
| | RHUS T., SABIN., SEC., SULPH., TUB. |
| HEAD DANDRUFF | CANTH., CARBN S. GRAPH., NAT M., PHOS., |
| | SULPH. |

TABLE 4
Abbreviations

Acon. (Aco.) - Aconitum
Napellus.
Aesc - Aesculus
Hippocastanum
Agar. - Agaricus
Muscarius.
Agn. (Ag c.) - Agnus
Castus.
Ail. - Ailanthus
Glandulosa.
All c. (Cep.) - Allium
Cepa.
Aloe (Alo.) - Aloe
Socotrina.
Alum. - Alumina
Ambr. (Amb.) - Ambra
Grisea.
Am c. - Ammonium
Carbonicum.
Am m. - Ammonium
Muriaticum.
Anac. - Anacardium
Orientale.
Ant c. - Antimonium
Crudum.
Ant t. - Antimonium
Tartaricum.
Apis (Ap.) - Apis
Mellifica.
Arn. - Arnica Montana.
Ars. - Arsenicum Album.
Ars i. - Arsenicum
Iodatum.
Arum t. - Arum
Triphyllum.
Aur. - Aurum
Metallicum.
Bad. - Badiaga.
Bapt. - Baptisia Tinctoria.
Bar c. - Baryta
Carbonica.
Bar m. - Baryta
Muriatica.
Bell. - Belladonna.
Bell p. - Bellis Perennis.
Benz ac. - Benzoicum
Acidum.
Berb. - Berberis Vulgaris
Both. - Bothrops
Lanceolatus
Bov. - Bovista.
Bry. - Bryonia Alba.
Bufo (Buf.) - Bufo Rana.
Cact. - Cactus Grandiflorus.
Calad. - Caladium Seguinum.
Calc. (Calc c.) - Calcarea
Carbonica.
Calc-i. (Calc io.) - Calcarea
Iodata.
Calc p. - Calcarea Phosphorica.
Calc s. - Calcarea Sulfurica.
Calen. (Calend.) - Calendula.
Camph. (Cam.) Camphora.
Cann i. (Cann.) - Cannabis
Indica.
Canth - Cantharis
Carb ac. - Carbolicum Acidum.
Carb an. (Carb-a.) - Carbo
Animalis.
Carb v. - Carbo Vegetabilis.
Carbn s. - Carboneum
Sulphuratum.
Caul. - Caulophyllum
Caust. (Caus.) - Causticum.
Cham. - Chamomilla.
Chel. - Chelidonium Majus.
Chim. - Chimaphila Umbellata.
Chin. - China Officinalis.
Chin ar. - Chininum
Arsenicosum.
Chlol. (Chl-hyd.) - Chloralum
Hydratum.
Cic. - Cicuta Virosa.
Clem. - Clematis Erecta.
Coca - Coca.
Cocc. (Cocl.) (Coccl.) -
Cocculus Indicus.
Coll. - Collinsonia Canadensis.
Con. - Conium Maculatum.
Cop. - Copaiva Officinalis.
Croc. - Crocus Sativus.
Crot h. - Crotalus Horridus.
Croto t. - Croton Tiglium.
Cupr. (Cup.) - Cuprum
Dig. - Digitalis Purpurea.
Dulc. - Dulcamara.
Elaps. - Elaps Corallinus.
Eup per. - Eupatorium
Perfoliatum.
Ferr. (Fer.) - Ferrum
Metallicum.
Ferr pic. - Ferrum Picricum.
Fl ac. (Flu ac.) - Fluoricum
Graph. (Grap.) - Graphites.
Guaj. - Guaiacum
Ham. - Hamamelis
Virginica.
Hell. - Helleborus Niger.
Helon. - Helonias Dioica.
Hep. - Hepar Sulphuris
Calcareum.
Hyos. (Hyo.) - Hyoscyamus
Niger.
Hyper. (Hypr.) - Hypericum
Iod. - Iodum.
Ip. - Ipecacuanha.
Iris (Iris v.) - Iris Versicolor.
Jug r. (Jugl.) - Juglans regia.
Kali ar. - Kali Arsenicosum.
Kali bi. - Kali Bichromicum.
Kali br. (Kali bro.) (Kali b.) -
Kali Bromatum.
Kali c. - Kali Carbonicum.
Kali i. (Kali io.) - Kali
Iodatum.
Kali s. - Kali Sulphuricum.
Kreos. (Kre.) - Kreosotum.
Lach. - Lachesis
Trigonocephalus.
Lappa (Lappa) - Lappa
Led. - Ledum Palustre.
Lyc. - Lycopodium
Clavatum.
Manc. - Mancinella.
Mang. - Manganum.
Meli. - Mellilotus
Officinalis.
Meny. (Men.) - Menyanthes.
Merc. (Merc.) (Merc viv.) -
Mercurius.
Merc c. (Mer cor.) -
Mercurius Corrosivus.
Merc I r. - Mercurius
Iodatus Ruber.
Mez. - Mezereum.
Mill. - Millefolium.
Mur ac. - Muriaticum
Acidum.
Nat ar. - Natrum
Arsenicicum.
Nat c. - Natrum
Carbonicum.
Nat m. - Natrum
Muriaticum.
Nat s. - Natrum
Sulphuricum.
Nicc. - Niccolum.
Nit ac. - Nitricum
Acidum.
Nux v. - Nux Vomica.
Ol j. - Oleum Jecoris
Aselli.
Op. - Opium.
Pæon. (Pae.) - Pæonia
Officinalis.
Par. - Paris Quadrifolia.
Petr. - Petroleum.
Ph ac. (Pho ac.) -
Phosphoricum Acidum.
Phos. (Pho.) -
Phosphorus.
Phyt. - Phytolacca
Decandra.
Plan. (Plant.) - Plantago
Major.
Plat. - Platinum
Psor. - Psorinum.
Puls. (Pul.) - Pulsatilla
Nigricans.
Rad br. (Radm.) -
Radium Bromatum.
Ran b. - ranunculus
Bulbosus.
Rhod. (Rho.) -
Rhododendron
Chrysanthum.
Rhus d - Rhus
Diversiloba
Rhus t. - Rhus
Toxicodendron.
Rumx. (Rum.) - Rumex
Crispus.
Ruta (Rut.) - Ruta
Graveolens.
Sabin. (Sabi.) - Sabina.
Sars. - Sarsaparilla.
Sec. (Sec c.) - Secale
Cornutum.
Sep. - Sepia Officinalis.
Sil. - Silicea.
Spig. (Spi.) - Spigelia
Anthelmia.
Spong. - Spongia Tosta
Staph. (Stap.) - Staphisagria.
Stram. (Stra.) - Stramonium.
Sul i. (Sul io.) - Sulfur Iodatum.
Sulph. (Sul.) - Sulphur.
Sul ac. - Sulphuricum acidum.
Symph. - Symphytum
Officinale.
Syph. - Syphilinum.
Tarent. (Tarn.) - Tarentula.
Tell. (Tel.) - Tellurium.
Teucr. (Mar.) - Teucrium
Marum Verum. Thlaspi
Thuj. (Thu.) - Thuja
Occidentalis.
Til. - Tilia Europœa.
Tub. (Tub.) - Tuberculinum.
Urt u. - Urtica Urens..
Valer. (Val.) - Valeriana
Officinalis.
Verat. (Ver a.) - Veratrum
Album.
Verat v. (Ver v.) - Veratrum
Viride.
Vinc. - Vinca Minor.
Viol t. (Vio t.) - Viola Tricolor.
Zinc. (Zin.) - Zincum
Metallicum.

The homeopathic composition or one or more ingredients thereof may be may be used to treat an ailment or injury for which it is known to be effective in accordance with any conventional homeopathic practice, such as that set forth in any homeopathic material medica, HPUS or other homeopathic treatment guide. Alternatively, the homeopathic composition may be used to treat an ailment or disease for which it has not been previously recognized as effective. As is the convention with homeopathic medicine, a single homeopathic composition may also be used to treat multiple different ailments or injuries. For example, a single homeopathic composition may be used to treat migraines, trauma to the eye, or sinusitis, as detailed in the homeopathic materia medica.

The homeopathic composition need not include counterirritant ingredients, and thus, does not rely upon the principal of producing a less severe pain to counteract a more intense pain. Consequently, the homeopathic composition may, but is not required to incorporate menthol, camphor, methyl salicylate, or trolamine salicylate. Additionally, the homeopathic composition may, but need not, include a chemical penetration enhancer such as alcohols, sodium lauryl sulphate, Pluronic F68, or similar substances.

The homeopathic composition is formulated to have a high potency and may be prepared in accordance with any method, such as that described in disclosed in the HPUS; Boericke, William, “Pocket Manual of Homeopathic Materia Medica,” B. Jain, 1995; Hahnemann, Samuel, “Materia Medica Pura,” 1830; or Schroyens, Frederik, “Synthesis Repertory 9,” Homeopathic Book Publishers, 2004. In an exemplary embodiment, the homeopathic composition of the present invention has a high potency of at least about 400 C. Alternatively, the homeopathic composition may have an exemplary potency of at least about 500 C, at least about 800 C, at least about 1M, at least about 2M, at least about 5M, at least about 10M, at least about 20M, at least about 50M, at least about 1 CM, at least about 2 CM, at least about DM, at least about MM, any intermediate potency therebetween, or any higher potency thereof, such as quintamillesimal (LM or Q) order potencies. Mixing of different potencies of the same or different homeopathic compositions and/or ingredients is also contemplated to be within the scope of the present invention. For example, a 1M arnica can be mixed with a 10M arnica or 10M ledum. Therefore, the homeopathic composition of the invention can be varied in terms of ingredients, potency or dosages as described herein.

Without wishing to be bound by theory, it is believed that the below described aqueous environment in which the homeopathic composition is maintained enables the composition to be formulated in and activated at high potencies. Unlike conventional gels which do not maintain a homeopathic composition in an aqueous environment and that are generally limited to a maximum potency of less than 200 C, the aqueous homeopathic composition of the present invention may be formulated at high potencies of about 400 C or more with beneficial results.

In an exemplary embodiment, exposure to direct sunlight, higher temperatures, volatile organic compounds, x-rays, and electromagnetic fields should be avoided during formulation, storing and shipping in order to prevent a change or neutralization in potencies. Preferably, the homeopathic formulation may be packaged in plastic, glass or other containers following HPUS, GMP's and all OTC regulations.

### 2. Delivery of the Homeopathic Composition in an Aqueous Environment

Another feature of the present invention is the delivery of the homeopathic composition in an aqueous environment. To accomplish this, the homeopathic composition may be dissolved in, suspended in or otherwise mixed with any aqueous medium that contains water molecules and is suitable for topical delivery. Exemplary aqueous media include water, water-containing azeotropic mixtures such as those containing alcohols, bodily fluids, herbal preparations containing water, any water based mixtures or combinations thereof. Preferably, the aqueous medium is a substance that does not irritate the skin, mucosal lining or other topical application surfaces. In one embodiment, the aqueous homeopathic composition may be formulated by mixing a homeopathic composition or a sucrose or lactose tablet homeopathic composition in water and/or a water-containing azeotropic mixture.

Optionally, the aqueous homeopathic composition may include one or more additives, provided that it includes a sufficient amount of water molecules to effectively treat an ailment or injury. Exemplary additives include gels, alcohols, herbal components, oils, water based mixtures or combinations thereof. In an exemplary embodiment, the homeopathic composition contains a sufficient amount of water to prevent the homeopathic composition from becoming substantially absorbed or substantially metabolized by the body before completion of the prescribed or predetermined treatment period.

The homeopathic composition of the present invention should be designed to maintain the homeopathic composition in an aqueous environment, by, for example, keeping the site of an ailment or injury wet with the homeopathic composition and at least some water for an extended period of time, at least for the prescribed treatment period. In an exemplary embodiment, the patient's bodily fluids may provide the required water and/or assist in maintaining the homeopathic composition in an aqueous environment during administration. For example, bodily fluids produced by open wounds or mucous and/or tears produced by the eye may produce a sufficient quantity of water molecules to maintain the homeopathic composition in an aqueous environment during treatment. Bodily fluids alone may include a sufficient quantity of water molecules to maintain the homeopathic composition in an aqueous environment sufficient for effective administration. For example, in instances wherein the homeopathic composition is administered to a mucosal membrane, the natural water-containing secretions of the mucosal membrane may maintain the homeopathic composition in an aqueous environment.

Without wishing to be bound by theory, it is believed that the aqueous environment containing water molecules provides superior therapeutic results. Studies have suggested that a unique structure may be formed when the homeopathic composition is diluted in water. Other studies have shown that the thermodynamic properties of water may be changed upon adding a homeopathic composition to water. A water deficiency in conventional gels and creams may limit both the potency levels and delivery periods of such homeopathic compositions.

### 3. Topically Administering the Aqueous Homeopathic Composition

After the high potency aqueous homeopathic composition is formulated, the composition may be topically administered to a patient. Specifically, the composition may be topically applied directly onto or one or more bodily surfaces, including any tissue, such as epithelial tissue, connective tissues, nervous tissues; any subcutaneous surface; muscles; organs; nerves; brain; arteriol; lymphatic; bone or combinations thereof. In an exemplary embodiment, the composition may be topically administered to the skin, eyes, ears, or mucosal lining of the nasal or anal cavity.

Topical administration involves the step of applying a high potency homeopathic composition directly to and maintaining contact with a bodily surface in an aqueous environment so that at least some of the homeopathic composition remains active and does not substantially become absorbed or otherwise metabolized until the end of the treatment period. As will be discussed below, a novel feature of this invention is maintaining the contact between a homeopathic composition in an aqueous environment and the bodily surface for a sufficient amount of time to effectively treat an ailment or injury. While not wishing to be bound by theory, it is believed that the direct contact between the bodily surface and the homeopathic composition while maintained in an aqueous environment extends the activity of and consequently increases the therapeutic effectiveness of the homeopathic composition. The homeopathic composition may be intermittently or continuously reapplied so as to provide either a continuous dosage or multiple dosages over time.

In an exemplary embodiment, the homeopathic composition may be administered directly to the site of an ailment or injury. In contrast to the conventional expectation that high potency topical compositions would be sub-lingually distributed and absorbed by the blood stream so as to provide a primarily systemic treatment and only limited, if any, localized therapy, when administered to the site of an ailment or injury and maintained in an aqueous environment, the topical homeopathic composition of the present invention is capable of delivering a direct localized treatment at high potency. In an exemplary embodiment, the homeopathic composition may be topically administered so as to completely cover the area of ailment or injury. Alternatively, smaller or larger topical administration areas are also contemplated.

Topical administration may also be accomplished using a delivery mechanism capable of both topical application and maintaining the homeopathic composition in an aqueous environment for an extended period of time. Exemplary delivery mechanisms may include absorbent continuous release mechanisms, such as gauze pads, paper towels, cotton balls, or bandages; a continuous release chemical means, such as a hydrogel, paste, foam, or tablet formulation, wherein the tablet is not intended to be swallowed but rather held in contact with a bodily surface such as the mucosal lining of the eye which provides the aqueous environment; a water reservoir, such as a foot bath or bathtub for containing an aqueous homeopathic composition and with which a bodily surface may be contacted, or partially or completely submerged, or any other means for topically delivering the homeopathic composition to and maintaining contact with a bodily surface in an aqueous environment over an extended period of time. In an exemplary embodiment, additional administrations of the homeopathic composition may be employed to reuse absorbent delivery mechanisms, such as a gauze pad, paper towel, cotton ball, or bandage, to maintain the homeopathic composition in an aqueous environment and extend the active period of the composition.

Alternatively, the delivery mechanism may be a continuous release chemical means that maintains the homeopathic composition in an aqueous environment, designed to release the homeopathic composition for an extended period of time and/or interact with a bodily surface, such as a mucosal membrane or bodily fluid, to continuously release the homeopathic composition. The continuous release chemical means, in use, must be able to maintain the homeopathic composition in an aqueous environment and in contact with a bodily surface for the duration of a treatment. In an exemplary embodiment, for homeopathic compositions that are intended to be topically administered to the skin, the delivery mechanism may include a means for maintaining the homeopathic product dissolved in an aqueous medium that contains a sufficient amount of water molecules so that not all of the homeopathic composition does not become absorbed, metabolized or evaporated in a time frame less than the requisite treatment period.

Optionally, topical application of the homeopathic composition need not, but may be combined with iontophoresis, electroporation, sonophoresis, phonophoresis, massage, or application of pressure to the site of administration. Furthermore the method of the present invention may be optionally performed in conjunction with any conventional homeopathic treatment, including any oral or other topical administrations.

In an exemplary embodiment, exposure to direct sunlight, high temperatures, volatile organic compounds, x-rays, and electromagnetic fields is preferably avoided during topical administration of the homeopathic composition.

### 4. Administering the Homeopathic Composition in an Aqueous Environment for an Extended Period of Time to Effectively Treat an Ailment or Injury

The method of the present invention may be used to effectively treat any ailment or injury, particularly acute and chronic ailments or injuries. For acute ailments or injuries, such as inflammation, pain, ecchymosis, boils, epistaxis, skin diseases, such as blisters, impetigo, tinea, herpes zoster, surgical injuries and herpes, the topically applied high potency homeopathic composition may be continuously administered until the ailment or injury is effectively treated. The homeopathic composition is most effective when it is administered relatively immediately after incurring the ailment or injury. To effectively treat inflammation, the homeopathic composition may be repeatedly administered until the acute inflammation phase is complete, wherein there is no increase in swelling upon removal of the homeopathic composition. To effectively treat pain, the homeopathic composition may be repeatedly administered until the pain is below a 1 of 10 on a visual analog scale (VAS). To effectively treat ecchymosis, the homeopathic composition may be repeatedly administered until removal of the remedy does not result in discoloration. To effective treat boils, the homeopathic composition may be administered until the boil either becomes resorbed or erupts. To effective treat epistaxis, the homeopathic composition may be administered until all blood flow from the nose has stopped for at least 5 minutes. To effectively treat skin issues, such as blisters, impetigo, tinea, herpes zoster and herpes, the homeopathic composition may be administered until the skin is healed and shows no visible signs of disease or other problems.

For chronic ailments or injuries, such as injuries to the ligament, tendon, bone, tissue, fistula, wart, anal fissure or ringworm, the homeopathic composition may be administered a few hours a day until the ailment is resolved. To effective treat an injury to ligament, tendon or bone, the homeopathic composition may be administered a few hours a day until the tissue is healed, as verified by MRI or X-ray. To treat external skin diseases, the homeopathic composition may be used daily until all signs of the ailment are gone, wherein the patient's hair stops falling out, the fistula, wart, anal fissure, ringworm or other ailment are completely gone.

Additionally, the method of the present invention is particularly useful for treating surgical injuries, including injuries incurred during, as a result of or in association with any surgical procedure, including removal or repair of circulatory, digestive, endocrine, lymphatic, integumentary, muscular, nervous, reproductive, respiratory, skeletal, urinary, sensory, or excretory systems. Exemplary surgical procedures may include abdominal surgery; abdominoplasty; adenoidectomy; amputation; angioplasty; appendicectomy; arthrodesis; arthroplasty; arthroscopy; biopsy; brain surgery; breast biopsy; cosmetic surgery, cauterization; cesarean section; cholecystectomy; circumcision; colon resection; colostomy; corneal transplantation; diverticulectomy; episiotomy; endarterectomy; fistulotomy; frenectomy; frontalis lift; fundectomy; gastrectomy; grafting; heart transplantation; hemorrhoidectomy; hepatectomy; hernia repair; hysterectomy; kidney transplantation; laminectomy; laparoscopy; laparotomy; laryngectomy; lumpectomy; lung transplantation; mammectomy; mammoplasty; mastectomy; mastoidectomy; nephrectomy; orchidectomy; pancreaticoduodenectomy; parathyroidectomy; prostatectomy; sigmoidostomy; sphincterotomy; splenectomy; thymectomy; thyroidectomy; tonsillectomy; tracheotomy; ulnar collateral ligament; reconstruction. Exemplary cosmetic surgical procedures, may include liposuction; liposculpture; rhinoplasty; rhytidectomy; blepharoplasty; sclerotherapy; vaginoplasty; phalloplasty; labiaplasty; abdominoplasty; chemical peels; surgical augmentations, implants or reductions, such as lip augmentation, chin augmentation or breast implants; mole removal; scar removal or repair; tattoo removal; skin resurfacing; dermabrasion; and collagen injections.

In another embodiment, the method of the present invention is used to treat deep tissue wounds in any portion of body, such as the epithelial tissue, connective tissue, muscles, nervous tissue, organ, nerve, brain, arteriol, lymphatic, or bone. The method may also be used to treat any injury or wound of, including deep tissue wounds of, as well as any ailment of the circulatory, digestive, endocrine, lymphatic, integumentary, muscular, nervous, reproductive, respiratory skeletal, urinary, sensory, or excretory systems.

The aforementioned effective treatment of an ailment or injury is achieved by administering a homeopathic composition in an aqueous environment for an extended period of time sufficient to provide a beneficial effect. This extended topical treatment period is a novel aspect of the present invention that has not been previously known to or recognized by the homeopathic community. Without wishing to be bound by theory, topically administering the homeopathic composition in an aqueous environment for an extended duration increases therapeutic effect.

The homeopathic composition can be administered intermittently or continuously for an extended period of time, including hours, days or weeks, as needed, to any bodily surface in order to effectively treat an ailment or injury. For topical administrations to a bodily surface, exemplary treatments may involve the topical application of a high potency homeopathic composition maintained in an aqueous environment continuously for about 30 minutes or more or intermittently for a total of 2 hours or more in a 24 hour period. Alternatively, exemplary treatments may be continuously or intermittently topically applied for about 2 hours or more in a 24 hour period, more preferably, about 4 hours or more in a 24 hour period, more preferably, about 8 hours or more in a 24 hour period, and most preferably, about 12 hours or more in a 24 hour period. In another exemplary embodiment, the treatment may be a continuous or intermittent topical application of a high potency homeopathic composition maintained in an aqueous environment for about 24 hours or more, preferably, about 36 hours or more, more preferably, about 48 hours or more, and most preferably, about 60 hours or more. In yet another embodiment, the treatment duration may be about 2 to about 12 hours, preferably, about 4 to about 12 hours, more preferably, about 6 to about 12 hours, and most preferably about 8 to about 12 hours. With respect to the aforementioned exemplary embodiments directed to intermittent treatment, the homeopathic composition may be applied in intervals of about 30 minutes or more, preferably, about 1 hour or more, more preferably, about 2 hours or more and most preferably, about 4 hours or more.

Without wishing to be bound by theory, it is believed that the homeopathic composition begins to produce therapeutic results within 60 seconds of topical administration. In general, the absorption of the homeopathic composition is limited by the formidable barrier provided by the skin, mucus membranes or other bodily tissue to which the homeopathic composition is applied. With respect to topical applications to the skin, it is generally recognized that short-term penetration occurs through the hair follicles and the sebaceous apparatus of the skin, while long term penetration occurs across cells. Initial absorption of small quantities of the homeopathic composition may occur within 60 seconds through the hair follicles and sweat glands. Because concentrations of homeopathic composition required to become therapeutic are very small it is expected that the product becomes therapeutic within 60 seconds of application.

The effective treatment of an ailment or injury is dependent upon the potency of a homeopathic composition, the duration of topical administration and the topical application to a surface area over which the homeopathic composition is applied. Each of these parameters in turn is dependant on and affected by the severity of the ailment as well as the body chemistry and tolerance of an individual patient. The more severe an ailment, the greater the potency, duration and/or applied surface area necessary to achieve effective treatment. In an exemplary embodiment, the method of the present invention may be tailored to the patient and injury to attain therapeutic effectiveness.

The following embodiments are provided as exemplary guidelines for treating various ailments and injuries using the method of the present invention. A skilled homeopath may make adjustments to these parameters, as necessary, within the scope of the present invention. Additionally, for purposes of the following exemplary embodiments, topical administration may be performed by applying an absorbent material, such as a gauze pad, contacted with the homeopathic composition and placed over the entire site of injury or ailment. The absorbent material should keep the site of injury or ailment continuously moist and in direct contact with the homeopathic composition throughout the duration of the treatment. The absorbent material may be removed and reapplied as needed. To prevent evaporation, the absorbent material may be covered with Tegaderm® or other plastic covering. To facilitate and expedite healing, the aqueous homeopathic composition is preferably applied to the site of injury or ailment as soon as possible after incurring the ailment or injury.

In an exemplary embodiment, the method of the present invention involves the localized and topical administration of a homeopathic composition including one or more of the compositions or ingredients listed in Table 1, in a potency of 400 C or more, to treat inflammation and maintaining the homeopathic composition in an aqueous environment in contact with an area of inflammation. The homeopathic composition is continuously administered until the acute inflammation phase is complete. The administration duration may be a continuous period of about 12 to about 48 hours. Subsequently, the homeopathic composition may be administered about 4 hours or more per day up to about 4 weeks until the sub-acute inflammation stage is complete.

In an exemplary embodiment, the method of the present invention may be used to effectively treat a patient for tissue damage, pain and/or any injury, such as a surgical injury or trauma. The method involves topically administering a homeopathic composition to the site of tissue damage, pain or surgical injury and maintaining the homeopathic composition in an aqueous environment in contact with the site for an extended period of time. The homeopathic composition may include Arnica montana, Bellis perennis, Calcarea phosphorica, Calendula, Hypericum perforatum, Ledum palustre, Rhus toxicodendron, Millefolium, Ruta graveolens, Symphytum officinale, Apis Mel, Cantharis, Urticartia Urens, Belladonna, Ferrum Metallicum, Staphasagria, Hepar Sulphericum, Euphatorium perfoliatum, Bryonia, Naturm Sulphericum, Calcarea carbonica, Hamamelis or combinations thereof, and may be formulated to have a potency of about 10M. The homeopathic composition may be continuously administered until the acute phase is complete. Exemplary administration duration may be a continuous period of about 12 to about 48 hours. Subsequently, the homeopathic composition may be administered in a potency of about 1M up to about 2 to about 24 hours a day for a period of up to about 4 weeks until the sub-acute stage is complete.

In an exemplary embodiment, the method of the present invention may be used to effectively repair tissue damage or treat pain and/or inflammation of soft tissue. Specifically, the method may be used to treat a patient for any surgical injury; treat a tear or injury to a ligament and/or tendon, such as tendonitis or a tear or injury of the anterior cruciate ligament (ACL); or treat an acute trauma, such as ecchymosis, sprain, concussion, muscle tear or strain, to repair soft tissue, alleviate pain and/or alleviate inflammation. The method involves topically administering a homeopathic composition formulated to have a potency of about 10M locally to the site of injury or ailment and maintaining the homeopathic composition in an aqueous environment in contact with the site. In one embodiment, the homeopathic composition may include a mixture of one or more of Arnica montana and/or Bellis perennis. In an alternative embodiment, the homeopathic composition may include a mixture of one or more of Arnica montana, Rhus toxicodendron, Ruta graveolens, and/or Ledum palustre. The homeopathic composition may be applied until the acute phase is complete, preferably for a period of about 12 to about 48 hours. Subsequently, a 1 M potency of the homeopathic composition may be applied until the sub acute phase is complete, preferably about 1 week or alternatively, up to about 4 weeks for about 2 to about 24 hours a day.

In an exemplary embodiment, the method of the present invention may be used to effectively repair tissue damage or treat pain and inflammation resulting from damage of nerve tissue, such as damage to the nerves in the fingertips, toes, genitals, spine tailbone, and/or eyeball. The method may involve localized application of a homeopathic composition including Hypericum perforatum formulated in a potency of at least 400 C or greater and maintaining the homeopathic composition in an aqueous environment for an extended period of time to provide effective therapeutic treatment. In another embodiment, the homeopathic composition may be formulated in a potency of about 1M and applied until the pain is eliminated or relieved. The homeopathic composition may be reapplied as needed to treat any reoccurrence of pain.

In an exemplary embodiment, the method of the present invention may be used to effectively repair tissue so and/or improve or facilitate healing of a wound, including broken skin injuries, such as cuts or scrapes; burns, such as chemical burns, temperature burns, or sunburns; or surgical incisions. The method may involve formulating a homeopathic composition including one or more of Calendula and/or Staphasagria in a potency of about 1M. The homeopathic composition may be applied directly to and/or around the wound until the wound is healed.

In an exemplary embodiment, the method of the present invention may be used to effectively repair bone tissue, such as bone tissue damaged by bone fractures, bone bruises, or trauma to the ocular region. The method may involve local application of a homeopathic composition including one or more of Symphytum officinale and/or Ruta graveonens formulated in a potency of about 1M. The homeopathic composition may be topically applied about 4 hours a day until the bone is healed.

In an exemplary embodiment, the method of the present invention may be used to effectively repair the tissue and/or treat pain and inflammation resulting from acute trauma to the head or scalp, such as would be incurred during a concussion. Alternatively, the homeopathic composition may be formulated in a potency of about 10M and applied until the acute phase is complete, preferably about 12 to about 48 hours. Subsequently, the 1 M homeopathic composition may be applied until the sub acute phase is complete, preferably about up to about 4 weeks for about 2 to about 24 hours a day.

In an exemplary embodiment, the method involves topical localized use of a homeopathic composition including one or more of the remedies listed in Table 2 in a potency of at least 400 C or greater maintained in an aqueous environment for an extended amount of time for acute conditions. A 1 M of the indicated homeopathic composition should be applied continuously locally until the problem is resolved. The effects are cumulative, thus if for patient comfort it is not possible to use the homeopathic composition continuously, the therapeutic effectiveness may be reduced. The entire area should be covered for maximum efficacy, if possible.

In an exemplary embodiment, the method of the present invention may be used to effectively treat boils. The method involves locally applying to the boil a homeopathic composition including Hep and Sil in a potency of about 1M and maintaining the homeopathic composition in an aqueous environment. The homeopathic composition may be continuously applied to the boil until it is resorbed or erupts.

In an exemplary embodiment, the method comprises topical localized use of a homeopathic composition including one or more of the remedies listed in Table 3 in a potency of at least 400 C or greater (at least 10⁻⁸⁰⁰ concentration) in an aqueous environment for an extended amount of time for chronic ailments. A 1 M potency of the indicated remedy should be applied locally 4-8 hours a day until the problem is resolved. The entire injured area should be covered for maximum efficacy, if possible.

In an exemplary embodiment, the method of the present invention may be used to effectively treat eczema or ringworm. The method involves locally applying a homeopathic composition as indicated in Table 3 to the site of the ailment in an aqueous environment. The homeopathic composition may be topically administered until eruption, preferably about 4 to about 8 hours per day. The absorbent material may be reapplied as necessary.

In an exemplary embodiment, the method of the present invention may be used to effectively treat hemorrhoids, fistulas or rectal fissures by locally applying a homeopathic composition as indicated in Table 3 and maintaining the homeopathic composition in an aqueous environment. The homeopathic composition may be applied to and cover an area around the anus. In an alternative embodiment, the composition may be formulated as a suppository which is released over an extended period of time, preferably over a period of about 4 to about 8 hours.

In an exemplary embodiment, the therapeutic may be used to treat strains, tears or other injuries to ligaments, such as the ligament of a knee. Upon application of the homeopathic composition having a potency of at least 400 C, the ligament will be effectively treated without requiring any or minimal surgical correction. To expedite healing, the knee may be kept in a straight leg brace to prevent bending. Optionally, the homeopathic composition may be inserted within the brace.

In an exemplary embodiment, the therapeutic may be used to treat blunt trauma to deep muscle tissue. The method involves topical application of a high potency aqueous based homeopathic composition including Bellis Perennis and Arnica having a potency of at least 400 C to the skin with an absorbent material for a period of about 8 hours. The aqueous homeopathic composition is effective for eliminating pain and inflammation of minor traumas and preventing ecchymosis associated with deep muscle bruises.

In an exemplary embodiment, the therapeutic may be used to treat minor abrasions. The method involves topical application of an aqueous based homeopathic composition including calendula formulated in a potency of at least 400 C and more to the minor abrasions for a period of about 4 hours a day. The aqueous homeopathic composition is applied to the area around the wound and may expedite the rate of healing by about 40%.

In an exemplary embodiment, the therapeutic may be used to treat bone fractures. The method involves topical application of an aqueous based homeopathic composition including symphytum and calc phos, or alternatively staphasagria formulated in a potency of at least 400 C to the skin surrounding a broken bone or fractured bone using an absorbent material. The treatment may substantially increase the fracture healing rate.

In an exemplary embodiment, the therapeutic may be used to treat surgical incision injuries. The method involves topical application of an aqueous based homeopathic composition including staphasagria in a potency of at least 400 C to the area around an incision for a period of about 4 hours a day. The treatment may substantially increase the healing rate by about 30%.

In an exemplary embodiment, the therapeutic may be used to treat cyst or boil that is sensitive to the touch. The method involves topical application of an aqueous based homeopathic composition including staphasagria in a potency of at least 400 C to the cyst or boil until it erupts or becomes resorbed over a period of about 24 hours.

The method of the present invention offers numerous advantages and unexpected benefits that are substantially more effective in treating an ailment or injury than conventional homeopathic therapies. The same remedies whose uses have been known for almost 200 years become substantially more effective for treating an ailment or injury, particularly a localized ailment or injury, when administered in accordance with the method of the present invention. The method substantially prevents, reduces the severity of, improves the condition of, expediting the healing of, cures, or combinations thereof any ailment or injury, including severe ailments or injuries. In contrast to most anti-inflammatories and analgesics, which have substantial side effects, the method of the present invention offers rapid and effective treatment without incurring harmful side effects. For example, relief from pain, inflammation, infection, or any symptom or condition of the ailment or injury may occur within about 30 seconds to about 2½ minutes from the first topical application of the high potency homeopathic composition of the present invention. Additionally, the therapeutic treatment may last for over 8 hours after application. Provided methods of use of homeopathic formulations according to the invention can also treat even difficult and/or chronic ailments, such as lyme disease pain, migraine headaches, etc., that are not normally considered topically treatable. Furthermore the treatment may effectively stabilize the ailment or injury on a long term to permanent basis without socially embarrassing odors, redness, stains, greasiness; or unpleasant physical sensations such as stinging, itching, burning, cooling sensations, irritations, drying of skin, or numbness, found in many, if not most, currently known topical analgesics or anti-inflammatory agents. Additionally, the degree of relief from pain and inflammation associated with injury (e.g., where a fully ruptured ACL may be painless and not swell) is not possible with current OTC or prescription medication without side effects. Oral aspirin and non-steroidal anti-inflammatory drugs dramatically increase the risk of peptic ulceration and renal failure when used chronically and increase the risk of drug interaction. Thus, because of unique topical effectiveness, use of provided methods of the invention may prevent many adverse internal reactions from unnecessary overuse of non-steroidal anti-inflammatory drugs.

### EXAMPLES

### Example 1

### Treatment for Bruising and Pain Associated with a Contusion

The method of the present invention was used to treat bruising and pain associated with contusion that was incurred when a boy fell from a 6 foot platform, caught his foot and fell face first into a pile of woodchips. His left eye took the brunt of the damage. An orange juice bottle was initially placed on the eye immediately after incurring the injury, but the eye remained quite painful. About 20 minutes after incurring the injury a large hematoma was apparent just beneath the eye in the shape of a woodchip. There were significant abrasions towards the outer canthi and above as well as below the eyebrow.

A first paper towel, approximately 4×4 in, soaked in a 10M Arnica and Ledum aqueous homeopathic composition that had been placed water to achieve the 10M potency, was topically administered around the boy's eye for about 20 minutes after incurring the injury. Within about 30 seconds of the topical application, the pain on and around the eye was relieved. When the first paper towel was removed, the pain returned within about 2 minutes. Upon reapplying the towel, the pain again was again alleviated. The first paper towel was topically applied for about 1 hour to about 2 hours, as needed, immediate after incurring the injury to relieve the pain

A second 1 sq in paper towel, soaked in the aqueous homeopathic composition, was then placed under the eye for about 8 hours to about 10 hours. Over the subsequent 36 hour period, the 1×1 paper towel was placed on the eye about 4 hours to about 8 hours each day, and the 4×4 paper towel was applied about 8 hours, to alleviate pain and reduce as well as expedite inflammation. After the first 48 hours, the 4×4 paper towel soaked in the mixture was placed on the eye 1-2 hours a day for the next 3 days

Generally, hematomas under the eye typically produce black eyes and swelling, and abrasions associated with injuries of this type typically cause bruising. However, about 3 days to about 7 days after incurring injury, there was no bruising, swelling or any discoloration after using the therapeutic method of the present invention. The large hematoma was healed in about 8 days, and the boy experienced no pain around or in the eye during and subsequent to treatment.

### Example 2

### Treatment for a Grade 2 ACL Tear

Due to its excellent vascularization, ACL tears typically swell to twice their normal size. This swelling generally lasts for about 2 weeks and causes extensive pain as well as severely restricts flexion and extension. According to the University of Washington department of orthopedics and sports medicine, an ACL injury results in significant swelling and decreased range of motion within a few hours of injury. The patient will have difficulty bearing weight. According to E-Medicine “A hemarthrosis almost always is present because of the vascular supply to the ACL”. In addition, range of motion is likely to be limited due to joint swelling.

An aqueous homeopathic composition of 10M Arnica, Ledum, Ruta and Rhus tox was applied for about 1 hour to a grade 2 partial ACL tear incurred by a woman within about 1 hour after incurring the injury. The administration was found to be effective for eliminating pain, eliminating swelling and facilitating healing. The aqueous homeopathic composition was prepared by placing two pellets each of 10M Arnica, Ruta, Ledum and Rhus tox in 1 pint of water. This aqueous homeopathic composition was sipped up to 10 times and applied externally. Multiple paper towels soaked in the aqueous homeopathic composition were placed around the knee to completely cover it front and back and to secure it with and ace bandage.

Subsequently, a second aqueous homeopathic composition was prepared by placing 10M Arnica pellets in 1 pint of water. This second composition was applied with a paper towel and effectively reduced swelling as well as eliminated pain of the surrounding soft tissue for about 20 minutes. At this time, ice was applied to the knee for about 1 minute causing pain. As the knee was very sensitive to ice, 2 pellets of 10M Rhus tox were added to the second aqueous homeopathic composition. The paper towel was subsequently resoaked and applied to the knee for about 2 hours, and the pain was eliminated within about 10 minutes.

The knee was examined within hours of the injury. Lachmans and anterior pivot shift was both positive, consistent with a grade 2 ACL tear. Minimal swelling was observed and weight bearing was possible with crutches. Due to the lack of swelling, the knee was extremely lax. A knee brace was used when walking to stabilize the knee.

During the night no homeopathic compositions were administered. Consequently, the next morning, the knee was swollen and painful, having a VAS pain level of about 3 of 10. Paper towels soaked in an aqueous homeopathic composition of 10M Rhus tox and Arnica that was prepared in water to achieve the 10M potency was applied around the knee, completely surrounding the joint. Pain and inflammation were completely eliminated within an hour. The aqueous homeopathic composition was then reapplied around the knee in the manner for at least about 20 hours over a period of 36 hours.

The knee remained painless with minimal swelling for the next few weeks. The patient was able to put weight on the knee without pain beginning the day after the injury. Additionally, the patient was able to walk with out crutches 3 days after the injury with a knee brace. A 1M Rhus tox and Arnica aqueous homeopathic compositions was topically administered to the knee for about 4 to about 8 hours a day over a period of 3 weeks and were found to be effective in promoting ligament and tendon healing.

Two weeks after the injury, the knee was stable enough to remove the knee brace. However, forgetting that the knee was damaged, the patient attempted to do a deep knee bend to pick something up and re-injured the knee causing immediate pain. The aqueous homeopathic composition of 10M Rhus tox and Arnica that was prepared in water to achieve the 10M potency was topically and continuously applied to the knee for 24 hours immediately after reinjury and for at least about 8 hours a day over a period of about one week. The knee remained painless even with weight bearing, however, the joint laxity could be felt.

Typically ACLs swell considerably when torn and are exceedingly painful. In addition, it is extremely rare for a grade 2 partial ACL tear to fully repair. In this case, pain and inflammation occurred only when the remedies were not used. In addition, the ACL completely heal so the patient was able to play tennis about 8 weeks after the injury. There have been no further complications. At this point, the patient plays tennis 3-4 times a week with no instability. The therapeutic method provided at least a 90% improvement in pain and inflammation resulting from the ACL rupture.

### Comparative Example A

### Cream Administration for Treating Bruising

For purposes of comparing the therapeutic results of Example 1, two cream based homeopathic compositions, a 1M and a 10M Rhus Tox, Ledum, Arnica and Ruta homeopathic composition, was prepared by incorporating the homeopathic composition in lanolin. The cream base was not designed to maintain the homeopathic composition in an aqueous medium. When applied to a bruise on the leg, no detectible improvement was detected as a result of using either cream based homeopathic compositions.

### Example 3

### Treatment for a Medial Meniscus Tear

After the ACL tear discussed in Example 2 was healed, the patient occasionally reported pain in a knee consistent with a MCL irritation or tear. About 2 years after the original ACL injury this pain became intense, suggesting a full tear. A 4×4 in paper towel soaked in an aqueous homeopathic composition of 1M Ruta, that was prepared in water to achieve the 1M potency, was applied to skin on the back of the knee. The aqueous homeopathic composition was topically administered on the knee for about 8 hours nightly for a period of about 2 weeks. The treatment was effective, relieving all pain. The pain did not return.

### Example 4

### Treatment for a Grade 3 ACL Rupture

An aqueous homeopathic composition of 10M Arnica, Ledum, Ruta and Rhus tox, that was formulated in water to achieve a 10M potency, was applied to a grade 3 ACL full rupture for about 48 hours after incurring the injury. Two pellets each of 10M Arnica, Ledum, Ruta, and Rhus tox were placed in 1 quart of water. Within about 1 hour of injury, paper towels soaked in the aqueous homeopathic composition were placed around the knee so that they entirely surrounded with knee. After about one hour, the homeopathic composition was exchanged for a second aqueous homeopathic composition of 10M Arnica, Rhus tox, and Ruta, that was formulated in water to achieve a 10M potency. This second aqueous homeopathic composition was applied for the about 48 hours and was effective in reducing pain and inflammation. Subsequently, a mixture of 1M arnica and rhus tox, that was formulated in water to achieve potency, was used at least about 4 hours a day for about two weeks. A 100M arnica and rhus tox aqueous homeopathic composition was also applied for the same duration. The treatment was found to be effective in promoting ligament and tendon healing.

Generally within about 2 hours of injury, a ruptured ACL is severely swollen and substantially restricts movement of the knee. An MRI of the knee, however, taken approximately 5 hours post injury showed only small/moderate joint effusion swelling according to the radiology report typically observed in ACL tears that have had a substantial period of time to heal.

The knee remained substantially painless throughout treatment, even upon bearing weight 3 days after incurring the injury. Only minor swelling was observed 1 and 3 days after incurring the injury so that it was difficult to distinguish between the injured and uninjured knee. The experienced orthopedic surgeon who examined the patient noted surprisingly only small to moderate joint effusion and retention of near full range of motion. Only moderate pain was reported during terminal flexion. After only 4 days, the patient, wearing a brace, was able to walk without crutches. The provided methods provide at least a 90% improvement in pain and inflammation in an ACL rupture.

### Example 5

### Treatment for ACL Reconstructive Surgical Injury

3 hours following ACL reconstructive surgery, a paper towel soaked with an aqueous homeopathic composition prepared by placing two pellets each of 10M Arnica and Hypericum in 1 quart of water was placed near the knee 2 hours after surgery. The composition was found to be effective in reducing pain and inflammation. Due to the post surgical dressing, the aqueous homeopathic composition could not be placed very close to the knee so it was placed on the lower thigh. Arnica is most commonly used for soft tissue repair, and Hypericum is used for nervous tissue repair. However, it appears that this did not address all of the injured tissue from the surgery. The knee was exceedingly painful, having a VAS level of 12 of 10. 2 tablets of hydrocodone were taken to eliminate the pain. Three hours after surgery, an aqueous homeopathic composition of 10M Rhus tox, Arnica and Hypericum, that was prepared in water to achieve the 10M potency, was applied to the knee using a 4×4 in gauze pad applied to the back of the knee for about 5 hours after surgery. Care was taken to avoid getting the sutures wet. Subsequently, the knee pain was reduced to about a VAS level of about 2 of 10. The pain stayed at a 2/10 VAS even after the hydrocodone wore off 6 hours later.

Although the knee had little pain, as a precaution, hydrocodone was taken again before going to bed, about 8 hours after the initial dose. The 10 M Rhus tox, Hypericum, and Arnica aqueous homeopathic composition was also applied to the skin by wetting a 4×4 in or larger gauze pad and securing it on the back of the knee for the next 48 hours. The application size was sometimes larger because the dressing would become soaked. The hydrocodone was not repeated, and no anti-inflammatories were taken at any time. After the application of the aqueous homeopathic remedies, the knee remained nearly painless, at a VAS level of 1-2 of 10, during the first 48 hours. In contrast, the anterior tibia, where no aqueous homeopathic composition was applied directly to the skin, had a VAS level of 4 of 10, and was particularly painful when the post surgical brace was used. This anterior tibia was more painful than the surgical site demonstrating the effective localized therapeutic treatment of the present invention.

The 10 M Rhus tox, Hypericum, and Arnica aqueous homeopathic composition was applied to the back of the knee at least 12 hours a day for the next two weeks and then reduced to about 4 hours a day for the following 2 weeks. The pain of the knee remained at 0-2 of 10 VAS for the duration of the treatment. Within 4 days of the surgery, the patient was able to bear weight on the leg and only refrained from walking without crutches under doctors orders.

13 days after the operation, the patient reported minimal pain and no erythema. The patient lacked about 70 degrees full extension and flexed to about 90-95 degrees. 28 days after surgery, the patient had full extension and lacked only about 7 degrees full flexion. There was no erythema and only mild effusion. The flexion and extension observed was within the top 90% of recovering surgical patients. Prior to beginning any physical therapy, the patient had 135 degrees of flexion in the right surgically repaired knee compared to 145 degrees in the left knee, with the right knee only lacking 5 degrees of extension.

The minimal pain and inflammation is particularly remarkable in the absence of anti-inflammatory medication as was the dramatic drop in pain from the first to the second day immediately after surgery, upon topical administration of the 10M Rhus tox, Hypericum, and Arnica aqueous homeopathic composition in the absence of pain medication. The high degree of flexion and extension is also significant in the absence of physical therapy and anti-inflammatory medication.

### Comparative Example B

### Oral Administration for Treating an ACL Tear

For purposes of comparing the therapeutic results of Examples 3-5, thirty seven patients receiving cruciate ligament replacement were orally administered 30× arnica, and thirty four other patients received a placebo. Two hours before the surgery the patients designed to receive arnica were administered an oral dose of 30× arnica. After surgery, an oral dose of 30× was given in 3 hour intervals and then subsequently administered about three times a day for up to 8 days. The study found there was no statistical difference between the pain levels experienced by the two groups of patients or the total amount of analgesics used, consequently the oral arnica administration was proven ineffective for treating an ACL tear. A study of the color change induced by administration of oral arnica demonstrated that there was no statistical difference between the placebo and oral arnica for days 1, 5, 7, and 10.

### Example 6

### Treatment for Rhinoplasty Surgical Injury

A young female patient's nose was surgically broken and reformed. Gauze was placed over the surgical site during surgery after surgical reconstruction. After surgery, a 2×2 in gauze pad was soaked in a 10M Arnica, Ruta, and Ledum aqueous homeopathic composition that was prepared by placing two pellets each of 10 M Arnica, Ruta, and Ledum in 4 oz of an ice water bath. The soaked gauze pad was folded, placed under a 2×2 in Tegaderm® adhesive, and topically applied immediately below the eye and as close to the nose cast as possible. The gauze pad was replaced on post operative day one, and permanently removed 48 hours post surgery. On post operative day 3, a 1M Arnica, Ruta and Ledum aqueous homeopathic composition that was prepared by adding 1M Arnica, Ruta and Ledum to 4 oz of water, was applied to a folded 2×2 in gauze pad that was subsequently placed on the patient's face, below the eye, every night for a period of 5 nights. The application was not adhered to the face and fell off if the patient moved.

The patient exhibited a 90% reduction in swelling and a 70% reduction in ecchymosis compared with similar rhinoplasty surgeries conducted by the same surgeon on patients who did not receive the aforementioned therapeutic treatment.

### Example 7

### Treatment of Rhinoplasty Surgical Injury

The same therapeutic procedure as described in example 6 was used on a middle aged woman who underwent rhinoplasty surgery. The patient demonstrated a 80% reduction in swelling and a 60% reduction in ecchymosis compared with similar rhinoplasty surgeries conducted by the same surgeon on patients who did not receive the aforementioned therapeutic treatment. The ecchymosis was limited to the inner eye area and there was very little swelling.

### Comparative Example C

### Oral Administration for Treating Rhinoplasty Surgical Injury

For purposes of comparing the therapeutic results of Examples 6-7, forty-eight patients who underwent primary rhinoplasty were randomized into three groups. The first group received 10 mg of methyl-prednisone, the second group received orally administered 1M arnica three times a day for 4 days, and the last group received neither agent. On the morning of the surgery, the patients took their first oral dosage of 1M arnica prior to the operation, and two doses of 1M arnica after the operation on the day of surgery and 3 doses of 12 C arnica each day afterwards. The degree of ecchymosis and edema were visually on a scale of 0-5 that was rated by a 3 person panel. 0 represented no ecchymosis, and 5 represented severe ecchymosis. Similarly, 0 represented no edema, and 5 represented severe edema. There was no statistical difference for the ecchymosis on day 2, however on day 8 the control group had a statistically significant increase of ecchymosis mean rating of 1.85 versus 0.92 (p<0.01). There was also a statistically significant difference in the mean rating of eczema 1.19 for the arnica group, 1.96 for the control (p<0.0001).

### Example 8

### Treatment of Rhytidectomy Surgical Injury

A female patient underwent a full face and neck lift. Gauze was placed over the surgical site during surgery immediately after surgical reconstruction. After surgery, a 4×4 in gauze pad was soaked in a 10M Arnica aqueous homeopathic composition, formulated by adding two pellets of 10 M Arnica to 4 oz of an ice water bath. The folded gauze pad was placed under a Tegaderm® adhesive and covered as much facial area as possible under the eye and over the cheek. Two additional 10M Arnica soaked 4×4 in gauze pads covered by Tegaderm® were placed on the neck. These dressings were removed and replaced after 24 hours. After the first 48 hours, the gauze pad was placed on the face, without adhesive, at night for the next 5 days. At times the patient indicated that the gauze pad fell off.

On the first day after surgery, there was no visible discoloration in the area where the aqueous homeopathic composition was used. The swelling was 90% improved over typical swelling, and the ecchymosis was 95% improved in the area where the gauze pads were placed. Some discoloration was observed behind the ear where the gauze pads were not administered, demonstrating the localized therapeutic effectiveness of the present invention. On day 3 the results were the same, 90% reduction in swelling and 95% reduction in ecchymosis. The patient returned to work about 5 days after surgery.

### Comparative Example D

### No Homeopathic Therapy for Treating Rhytidectomy

For purposes of comparing the therapeutic results of Example 8, the same patient had had the same procedure performed 10 years prior to the rhytidectomy described in Example 8 by the same physician. In this instance, the patient suffered severe ecchymosis that prevented her from returning to work for 3 weeks.

### Example 9

### Treatment for Rhytidectomy and Chin Implant Surgical Injury

A full face and neck lift as well as a chin implant was performed on a patient. The same therapeutic procedure described in Example 8 was used in the present instance, with the exception that the aqueous homeopathic composition was also topically applied to the chin. On post operative days 1, 3 and 7, the areas where the aqueous homeopathic composition was directly applied had minimal to no discoloration, showing about a 90% improvement and about 90% reduction in swelling. Significant discoloration was observed above the chin and slight discoloration was observed behind the ear, both regions where the mixture was not directly applied.

### Example 10

### Treatment for Rhytidectomy Surgical Injury

Rhytidectomy was performed on two patients, who were subsequently treated with the same therapeutic method as disclosed in Example 8. Both patients showed better than 90% improvement in ecchymosis and swelling on post operative day 1 and 3, with virtual elimination of discoloration by post operative day 7.

### Comparative Example E

### Oral Administration for Treating Rhytidectomy Surgical Injury

For purposes of comparing the therapeutic results of Examples 8-10, twenty-nine patients that underwent rhytidectomy were treated perioperatively with either oral homeopathic arnica or a placebo. On the morning of the surgery, the patients were orally administered their first oral dosage of 1M arnica prior to operation, then two doses of 1M arnica was orally administered after the operation the day of surgery and 3 doses of 12 C each day afterwards. The degree and area of eccymosis was analyzed using computer color analysis. There was no statistically significant difference in the ecchymosis between the control group and the placebo group. All patients showed a steady recovery with most of the ecchymosis resolved within 2 weeks. There was no statistically significant difference in the level of ecchymosis between the control and placebo group on post operative day 11. There was, however, a statistically significant reduction in the area of ecchymosis for orally administered arnica, which had a 9.5% and 29.10% reduction in the area of eccymosis on post operative days 1 and 7, respectively. By contrast, Examples 8-10 all showed at least a 90% reduction of ecchymosis.

### Comparative Example F

### Low Potency Therapy for Treating Rhytidectomy Surgical Injury

For purposes of comparing the therapeutic results of Examples 8-10, the effectiveness of a low potency 30 C topically administered aqueous based homeopathic composition for treating rhytidectomy surgical injuries was studied. A full face rhytidectomy was performed on a patient. Gauze was placed over the surgical site during surgery and after surgical reconstruction. After surgery, a 2×2 gauze pad was soaked in a 30 C arnica aqueous homeopathic composition, formulated by adding two pellets each of 30 C arnica to 4 oz of an ice water bath. The gauze was folded, and placed under a Tegaderm® adhesive, and topically administered so as to cover as much facial area as possible under the eye and over the cheek. Two additional 2×2 in gauze pads covered by Tegaderm® were placed on the neck. These dressings were removed and replaced after 24 hours. There was no detectable improvement in the discoloration or swelling.

### Example 11

### Treatment for Upper and Lower Blepharoplasty Surgical Injury

Upper and lower blepharoplasty surgery was performed on a male patient. Three hours after surgery, a 2×2 gauze pad was soaked in a 10 M Arnica, Ruta, and Ledum aqueous homeopathic composition, formulated by adding two pellets each of 10 M Arnica, Ruta, and Ledum to a 4 oz ice water bath. The gauze pad was folded, placed under a 2×2 in Tegaderm® adhesive, and topically applied immediately below the eye. Although the patient was instructed to topically apply the 2×2 gauze at night to the upper lid in intervals of 15 minutes, i.e. 15 minutes on and 15 minutes off, the patient was not consistent in performing the therapeutic method. The gauze pad was replaced on the first post operative day and removed 48 hours post surgery. After the first 48 hours, each night for the following 5 days, the gauze pad was placed on the eyes, without adhesive. At times the patient indicated that the gauze pad fell off. The patient showed a 30% reduction in ecchymosis and 80% reduction in swelling to the top and bottom eye on post operative days, 1, 3 and 7.

### Example 12

### Treatment for Lower Blepharoplasty Surgical Injury

Lower blepharoplasty was performed on a patient. During surgery, a 10 M Arnica, Ruta, and Ledum aqueous homeopathic composition formulated by adding two pellets each of 10 M Arnica, Ruta, and Ledum to 4 oz of an ice water bath, was topically applied to the reconstructed tissue as each portion of the reconstructed tissue was completed. After surgery a 2×2 in gauze pad was soaked in the aqueous homeopathic composition, folded, and placed under a 2×2 in Tegaderm® adhesive and topically applied immediately below the eye. The 2×2 in gauze pad was replaced on post operative day one and removed 48 hours post surgery. After the first 48 hours, the gauze was topically applied on the eyes, without an adhesive, at night for the next 5 days. At times the patient indicated that the gauze pad fell off. The patient showed a 80% reduction in ecchymosis and 80% reduction in swelling to the top and bottom of the eye on post operative days, 1, 3 and 7.

### Example 13

### Treatment for Upper and Lower Blepharoplasty Surgical Injury

Upper and lower blepharoplasty was performed on an elderly male patient. During surgery, a 10 M Arnica, Ruta, and Ledum aqueous homeopathic composition formulated by adding two pellets each of 10 M Arnica, Ruta, and Ledum to 4 oz of an ice water bath, was topically applied to the reconstructed tissue as each portion of the reconstructed tissue was completed. A 2×2 gauze soaked with the aqueous homeopathic composition to the eye in intervals of 15 minutes, i.e. 15 minutes on and 15 minutes off, for 48 hours. No adhesives were used and the gauze fell off at night. After the first 48 hours the mixture was placed on the eyes, without adhesive, at night for the next 5 days. At times the patient indicated that the gauze pad fell off. This patient showed a 80% reduction in ecchymosis and 90% reduction in swelling to the top and bottom of the eyes on post operative days 2-7. Some yellowing of the skin was observed on post operative days 2-5.

### Example 14

### Treatment for Upper and Lower Blepharoplasty Surgical Injury

The same method used in Example 13 was used on an elderly female patient having elective upper and lower blepharoplasty surgery. The patient reported very little discoloration and swelling and showed a 90% reduction in ecchymosis and 90% reduction in swelling at the top and bottom of the eyes.

### Example 15

### Treatment for Upper Blepharoplasty Surgical Injury

Upper blepharoplasty was performed on a patient. During surgery, a 10 M Arnica, Ruta, and Ledum aqueous homeopathic composition formulated by adding two pellets each of 10 M Arnica, Ruta, and Ledum to 4 oz of an ice water bath, was topically applied to the reconstructed tissue as each portion of the reconstructed tissue was completed. The aqueous homeopathic composition was applied for approximately one hour after surgery. On post operative days 1, 3, 7 no discoloration or swelling was noticed, representing a 98% improvement.

### Example 16

### Treatment for Liposuction Surgical Injury

A female patient underwent liposuction of the left and right flanks. A chux pad was soaked in aqueous homeopathic composition of 10M arnica and applied to the surgical area on the right side of her body. An identical chux pad was soaked in plain water and applied to the left surgical area. Saran wrap and a compression garment were applied over the chux pads on both sides. Approximately 24 hours later, the chux pads were removed and each side was photographed. The right side of the body showed a reduction in discoloration by at least 60% when compared to the left, particularly in the upper area. In addition, the chux pad was inadvertently moved when applying the compression garment and the entire surgical area was not covered. A distinct line was evident on the right side between where there was no ecchymosis, corresponding to the exact location of the application of the chux pad, and where there was sever ecchymosis, approximately 60% worse, where no application was made, adjacent to the treated tissue. There area immediately outside of the homeopathic application pad was bruised 60% more than the area treated using the chux pads.

1-50. (canceled)

51. A therapeutic method comprising the steps of maintaining a homeopathic composition having at least one homeopathic active agent at a potency of at least 400 C in an aqueous environment in continuous contact with a bodily surface for a period of time sufficient to effectively treat trauma, ailment or disease.

52. The method of claim 51, wherein said homeopathic composition has a potency of at least 1M or a potency of at least 10M.

53. The method of claim 51, wherein the homeopathic composition comprises one or more active agents selected from the group consisting of Arnica montana, Bellis perennis, Calcarea phosphorica, Calendula, Hypericum perforatum, Ledum palustre, Rhus toxicodendron, Millefolium, Ruta graveolens, Symphytum officinale, Apis Mel, Cantharis, Urticartia Urens, Belladonna, Ferrum Metallicum, Staphasagria, Hepar Sulphericum, Euphatorium perfoliatum, Bryonia, Naturm Sulphericum, Calcarea carbonica and Hamamelis.

54. The method of claim 51, wherein the homeopathic composition comprises Arnica Montana, Hypericum perforatum, Ledum palustre, Rhus toxicodendron and Ruta Graveloens.

55. The method of claim 51 wherein the homeopathic composition is administered to a subject for an injury of the anterior cruciate ligament.

56. The method of claim 51, wherein said homeopathic composition is continuously topically administered to said bodily surface for a period of at least 30 minutes.

57. The method of claim 51, wherein said homeopathic composition is maintained in contact with said bodily surface intermittently for a total of 2 hours or more at a time.

58. The method of claim 51, wherein said bodily surface is skin.

59. The method of claim 51, wherein said method effectively treats an injury associated with a plastic surgery procedure selected from the group consisting of: liposuction; liposculpture; rhinoplasty; rhytidectomy; blepharoplasty; sclerotherapy; vaginoplasty; phalloplasty; labiaplasty; abdominoplasty; chemical peels; surgical augmentations; surgical implants; surgical reductions; mole removal; scar removal or repair; tattoo removal; skin resurfacing; dermabrasion; and collagen injections.

60. The method of claim 51, wherein said method effectively treats a deep tissue wound in a portion of body selected from the group consisting of: epithelial tissue, connective tissue, muscles, nervous tissue, organ, nerve, brain, arteriol, lymphatic and bone.

61. The method of claim 51, wherein said method effectively treats a deep tissue wound of the circulatory, digestive, endocrine, lymphatic, integumentary, muscular, nervous, reproductive, respiratory, skeletal, urinary, sensory, or excretory systems.

62. The method of claim 51, wherein said homeopathic composition comprises a homeopathic active agent at a potency of at least 400 C contained within an absorbent material.

63. A homeopathic composition comprising at least one active homeopathic agent having a potency of greater than 400 C in an aqueous environment sufficient to maintain the activity of the homeopathic agent.

64. The homeopathic composition of claim 63, having a potency of 1M or a potency of 10M.

65. The homeopathic composition of claim 63, comprising at least one active homeopathic agent selected from the group consisting of Arnica montana, Bellis perennis, Calcarea phosphorica, Calendula, Hypericum perforatum, Ledum palustre, Rhus toxicodendron, Millefolium, Ruta graveolens, Symphytum officinale, Apis Mel, Cantharis, Urticartia Urens, Belladonna, Ferrum Metallicum, Staphasagria, Hepar Sulphericum, Euphatorium perfoliatum, Bryonia, Naturm Sulphericum, Calcarea carbonica and Hamamelis.

66. The homeopathic composition of claim 63, comprising Arnica Montana, Hypericum perforatum, Ledum palustre, Rhus toxicodendron and Ruta Graveloens.

67. The method of claim 51, wherein said method effectively treats an ACL rupture in a knee of a leg without surgery by using the homeopathic composition and placing the leg in a straight leg brace.
